(19) United States
(12) Patent Application Publication
(10) Pub. No.: US 2005/0261713 A1
(43) Pub. Date: Nov. 24, 2005

(54) Methods and devices for producing turbulence in vascular blood flow

(76) Inventors: Ali H. Hassan, Palo Alto, CA (US); Brian K. Courtney, Toronto (CA); Peter J. Fitzgerald, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/115,969

(22) Filed: Apr. 26, 2005

Related U.S. Application Data
(60) Provisional application No. 60/565,970, filed on Apr. 27, 2004.

(51) Int. Cl. A61M 29/00

(52) U.S. Cl. 606/157

(57) ABSTRACT

Methods and devices for producing turbulence in vascular blood flow are provided. In practicing the subject methods, a blood flow modulator is positioned on an external site of a blood vessel at a location in relation to, e.g., at least proximal to a branched vascular site in a manner sufficient to produce turbulence in blood flow at a target site, e.g., at or immediately proximal to the branched vascular site. Representative blood flow modulation devices that find use in the subject methods are devices that can be positioned on an external site of a blood vessel to produce an annular structure around the blood vessel, where the annular structure includes an inner surface protuberance of sufficient dimensions to produce turbulence in the blood vessel. Also provided are kits and systems for practicing the subject methods. The subject methods and devices find use in a variety of applications, including applications to reduce the risk of emboli entering branch blood vessels from a main blood vessel.

### CROSS-REFERENCE TO RELATED APPLICATIONS

Pursuant to 35 U.S.C. § 119 (e), this application claims priority to the filing date of the U.S. Provisional Patent Application Ser. No. 60/565,970 filed Apr. 27, 2004; the disclosure of which is herein incorporated by reference.

### FIELD OF THE INVENTION

The field of this invention is the treatment of vascular conditions.

### BACKGROUND OF THE INVENTION

Various vascular and/or cardiac procedures, such as the positioning of a stent graft or a bypass graft, stenting, angioplasty, valve repair, or procedures involving temporary halting or alteration of the cardiac rhythm, such as cardioversion, can create and/or release particles or emboli from the wall of an artery or the chambers of the heart. The dislodged emboli become entrained in the blood stream flowing through the lumen of the artery. If allowed to remain in the blood flow, the emboli are carried through the vascular system until they lodge in a blood vessel thereby forming a blockage or embolism. Depending upon the size and/or volume of the emboli and where in the vascular system they lodge, the consequences of an embolism can be extremely serious, resulting, for example, in the sudden cessation of blood flow to an extremity, an organ, such as a kidney, the brain or the heart.

Emboli with the potential to cause stroke can also originate from areas of injury or pathology in the cardiovascular system. Atrial fibrillation is an increasingly common condition in which the left atrium does not contract in an organized fashion. This disorganized contraction pattern creates regions in the left atrium where thrombi can form and become emboli. Another source of emboli is when people have a patent foramen ovale. Normally, thrombi that form in the systemic venous circulation, such as in the veins of the legs, are filtered by the vascular bed of the lung before returning to the left sided chambers of the heart. However, a patent foramen ovale, as well as other forms of shunts between the left and right-sided circulation systems, can allow thrombi in the legs to enter the left side of the heart without being filtered by the lungs. Once in the left sided circulation, these thrombi can cause strokes or other consequences of ischemia to other arterial vascular beds. Yet another source of embolic thrombi cause be mechanical valves that are used to surgically replace cardiac valves such as the aortic and mitral valves. Patients with mechanical valves are usually subsequently anti-coagulated with medicines such as warfarin and/or heparin to reduce the likelihood of thrombi being generated as a result of the thrombogenic surfaces and flow patterns introduced by the mechanical valves.

In certain instances, debris that is carried by the bloodstream to distal vessels of the brain can cause these cerebral vessels to obstruct/impair blood flow, resulting in a stroke, and in some cases, death. The term “stroke” is used to describe a medical event whereby blood supply to the brain or specific areas of the brain is restricted or blocked to the extent that the supply is inadequate to provide the required flow of oxygenated blood to maintain function. The brain becomes impaired, either temporarily or permanently, with the patient experiencing a loss of function such as sight, speech or control of limbs. There are two distinct types of stroke, hemorrhagic and embolic, the latter of which being caused by emboli present in the vessels of the brain.

In view of the above, there is clearly a need for a method that safely deviates debris from entering brain-bound branches of the vascular system, thereby not interfering with treatment instruments located within the vascular system. The present invention satisfies this need.

Relevant Literature

Patent Documents of interest include: (1) U.S. Pat. Nos. 5,211,649; 5,383,882; 5,417,702; 5,618,307; 5,569,274; 5,628,307; 5,707,378; 5,766,218; 5,928,253; and (2) published PCT application WO 00/32113.

### SUMMARY OF THE INVENTION

Methods and devices for producing turbulence in vascular blood flow are provided. In practicing the subject methods, a blood flow modulator is positioned on an external site of a blood vessel at a location in relation to, e.g., at least proximal to, a branched vascular site in a manner sufficient to produce turbulence in blood flow at a target location, e.g., at or immediately proximal to the branched vascular site. Representative blood flow modulation devices that find use in the subject methods are devices that can be positioned on an external site of a blood vessel to produce an annular structure around the blood vessel, where the annular structure includes an inner surface protuberance of sufficient dimensions to produce turbulence in the blood vessel. Also provided are kits and systems for practicing the subject methods. The subject methods and devices find use in a variety of applications, including applications to reduce the risk of emboli entering branch blood vessels from a main blood vessel.

### BRIEF DESCRIPTION OF THE FIGURES

FIG. 1 provides a depiction of a branched vessel and blood flow therein.

FIGS. 2A and 2B provide representations of a blood flow modulation device according to a first embodiment of the invention.

FIGS. 3A and 3B provide representations of a second blood flow modulation device embodiment of the invention.

FIGS. 4A to 4C provide representations of the blood flow modulator device of FIG. 2 positioned around the representative vessel of FIG. 1.

FIGS. 5A to 5D show various representations of the use of a device according to the present invention to reduce the flow of blood emboli into the brachiocephalic trunk from the aortic arch.

FIGS. 6A and 6B provide representations of a device having a semi annular configuration in an open and closed configuration, respectively.

### DESCRIPTION OF THE SPECIFIC EMBODIMENTS

Methods and devices for producing turbulence in vascular blood flow are provided. In practicing the subject methods, a blood flow modulator is positioned on an external site of a blood vessel at a location in relation to, e.g., at least proximal to a branched vascular site in a manner sufficient to produce turbulence in blood flow at a target site, e.g., at or near the branched vascular site. Representative blood flow modulation devices that find use in the subject methods are devices that can be positioned on an external site of a blood vessel to produce an annular structure around the blood vessel, where the annular structure includes an inner surface protuberance of sufficient dimensions to produce turbulence in the blood vessel. Also provided are kits and systems for practicing the subject methods. The subject methods and devices find use in a variety of applications, including applications to reduce the risk of emboli entering branch blood vessels from a main blood vessel.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Prior to reviewing the subject methods in more detail, a review of blood flow patterns is provided. The typical pattern of flow inside a blood vessel is laminar, streamlined and smooth. Blood usually flows in the shape of a parabolic curve with the fastest velocity in the center of the tube and slowest velocity along the internal or luminal surface of the vessel due to the friction of the blood with the sides of the walls of the vessel. Particles suspended in laminar-flowing blood follow the streamlines of the flow, and are distributed according to velocity, which is a function of particle size. As such, larger size particles are found in those layers of fluid near the luminal surface of the vessel, while smaller size particles are found nearer the center of the vessel. As large size particles suspended in the laminar-flowing blood follow the streamlines along the internal or luminal surface, they are inclined to divert into side branches.

In turbulent flow, particles can move in any direction—only the mean velocity and direction can be defined. Turbulence occurs when flow reaches a critical velocity and the orderly arrangement of particles, found in laminar flow, becomes disrupted. Random movement of these particles results in the transfer of energy between colliding molecules, as well as transient pressure fluctuations. The result of such flow disruption is that large particles suspended in the flow gain kinetic energy, and escape diversion into side branches. The inventors have realized that turbulent flow can be caused by the overall shape of the blood vessel or disturbances in the surface relief of the vessel, as well as by external vibration/energy applications to a desired vascular site, as described in greater detail below.

In further describing the subject invention, the subject methods and devices are described first in greater detail, followed by a review of representative applications therefore, as well as a review of representative systems and kits according to the present invention.

Methods and Devices

As summarized above, the subject invention provides methods of producing turbulence in blood flow in a vessel. By producing turbulence in blood flow in a vessel is meant changing or disrupting the normal laminar blood flow pattern in a blood vessel, and specifically an arterial blood vessel, e.g., the aorta or other arterial blood vessel. As such, when turbulence is produced by the subject methods, the normal laminar blood flow pattern in a blood vessel is disrupted or altered so as not to be present. Instead, a disorganized or random blood flow pattern is produced in the blood vessel, which produced blood flow pattern can only be characterized in terms of mean velocity and direction.

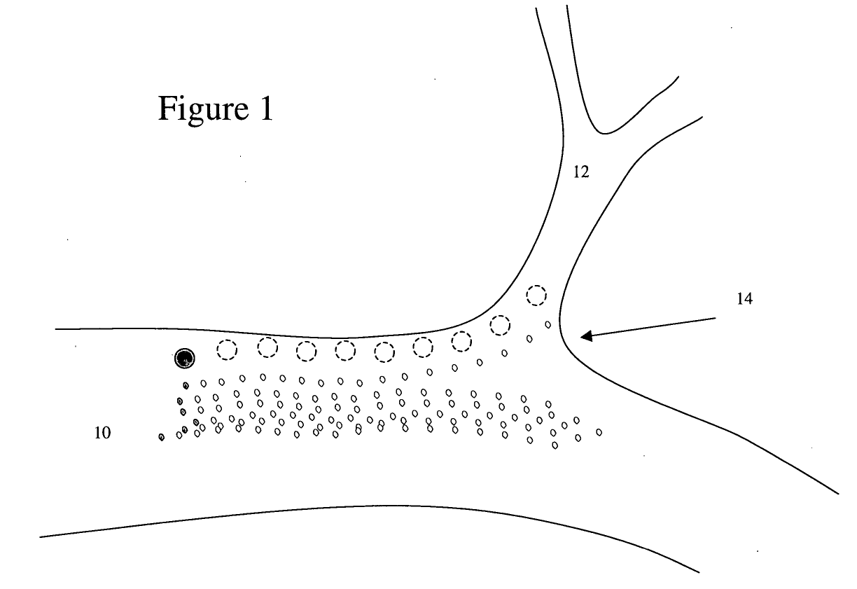

In practicing the subject methods, turbulence is produced in a blood vessel at a target location, e.g., at or proximal to a blood vessel branch point. In other words, turbulence is produced at a vascular site that is at least proximal to, e.g., in which, a main blood vessel opens into at least one branch or side blood vessel. Accordingly, the blood vessel branch point is a site or location in a blood vessel system, in which blood can either continue flowing through the blood vessel or can flow into the branch vessel. A representative branch point is shown in FIG. 1A, where main blood vessel 10 opens into branch vessel 12 at branch point 14.

Conveniently, turbulence is produced by positioning or placing a blood flow modulator at an external site of the blood vessel in which turbulence production is desired. In other words a blood flow modulating device is located on an outer surface region or area of the blood vessel in which the laminar blood flow is to be disrupted. In representative embodiments, the external site or location at which the device is positioned is one that is near or adjacent the branch point at which the production of turbulence is desired. By near or adjacent is meant that the external site at which the modulator device is positioned is less than about 100 mm, such as less than about 50 including less than about 10 from the branch point of the vessel in which the production of turbulence is desired, where in many embodiments the device is positioned from about 100 to about 50 mm, such as from about 30 to about 1 mm from the branch point.

The blood flow modulating device that is positioned at the external site, as described above, is one that, upon placement at the target external site of the blood vessel, produces the desired turbulence in the vessel. In representative embodiments, the device is one that changes the structure of the inner surface of the vessel, i.e., alters the inner surface relief of the vessel, in a manner sufficient to produce the desired turbulence in the vessel.

A representative device according to the subject methods is a device that, upon placement at the target external vessel site, produces an annular structure around the vessel, where the annular structure causes the desired change or alteration in internal vessel surface relief and concomitant production of turbulence in the vessel. The annular structure of this type of representative embodiment is a ring-like structure that may or may not be a complete ring, such that when placed around the vessel, the ring-like structure may or may not include at least one gap in the ring. A feature of these embodiments is that the inner surface of the annular structure that is positioned around the blood vessel of interest is one that includes at least one protuberance, i.e., a structure that projects from the inner surface. The protuberance is one that has sufficient dimensions to result in the desired change or alteration in the vessel inner surface relief, as described above. In certain representative embodiments, the protuberance is one that rises above the plane of the inner surface of the annular structure by at least about 0.05×radius, such as by at least about 0.10×radius, including by at least about 1.50×radius. The protuberance may be static or size adjustable. For example, in certain embodiment the protuberance has a shape that cannot be changed or altered, but is rigid or static. In yet other embodiments, the protuberance has a shape that can be changed or altered, e.g., by using an inflatable protuberance having an interior volume or region that can be filled with a fluid in a manner sufficient to adjust the protuberance to desired dimensions. Size adjustment, e.g., via inflation, could be prior to or after positioning the device about a target structure, as desired, including an extended period of time, e.g., days, weeks, months, following placement of the structure. Inflation and/or deflation can be achieved using any convenient protocol, e.g., via a syringe coupled to a fluid conveyance structure in fluid connection with the chamber within the protuberance, where the fluid conveyance structure may be accessed via a sealable injection port.

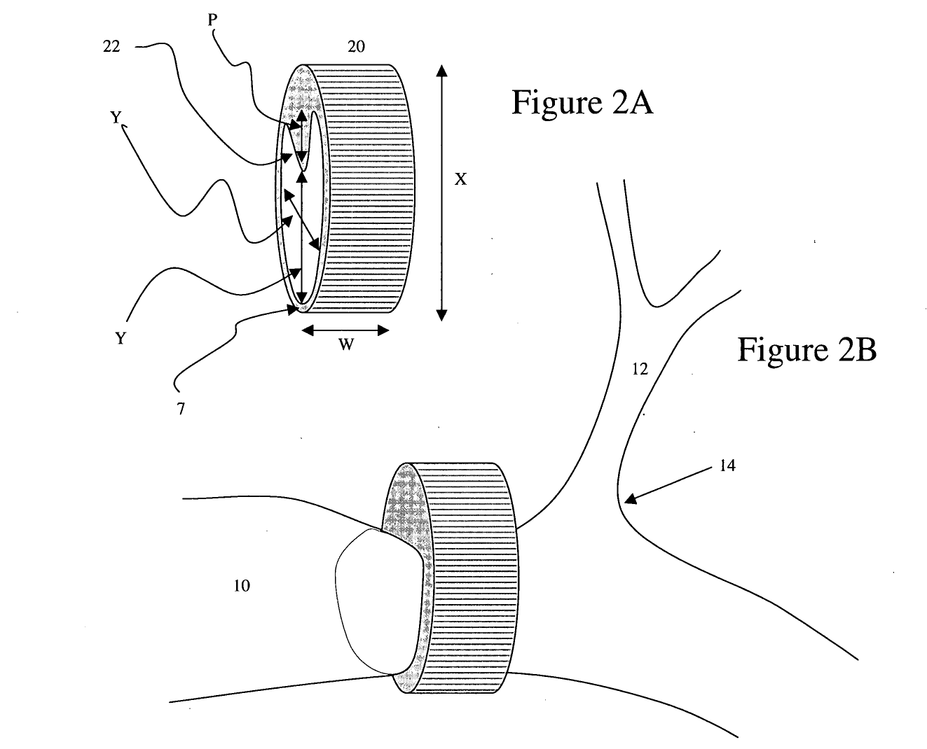

A representative annular structure produced by devices according to these embodiments of the subject invention is shown in FIGS. 2A and 2B, where device 20 is shown as a full ringlike structure having inner surface protuberance 22. Annular structure is configured or dimensioned to be positioned around an arterial vessel. As such, the outer diameter of the structure (“X” in figures) typically ranges from about 70 mm to about 1 mm, such as from about 60 mm to about 10 mm, including from about 50 mm to about 20 mm. The minimum inner diameter of the structure (Y in the figure) typically ranges from about 65 mm to about 0.5 mm, such as from about 55 mm to about 5 mm, including from about 45 mm to about 17 mm. The wall thickness of the structure (7 in the figure) in the region that is not occupied by protuberance 22 typically ranges from about 10 mm to about 0.1 mm, such as from about 5 mm to about 1 mm, including from about 3 mm to about 1 mm. The structure has a width (W in the figure) ranging from about 50 mm to about 1 mm, such as from about 40 mm to about 10 mm, including from about 30 mm to about 20 mm. Protuberance 22 typically rises above the inner surface of the structure a distance (p) that may range from about 50 mm to about 0.5 mm, such as from about 40 mm to about 1 mm, including from about 30 mm to about 5 mm. While the above-described representative embodiment includes a single protuberance, structures with two or more, i.e., a plurality of, protuberances, may be employed. The more than one protuberance can be arranged in any desired manner in a given cross-section, including eccentric, bicentric, multicentric, and concentric, depending on the flow path modulation desired to be produced. The protuberances can be arranged adjacent to each other in a number of different configurations, including radially about the target vessel, longitudinally along the target vessel, etc.

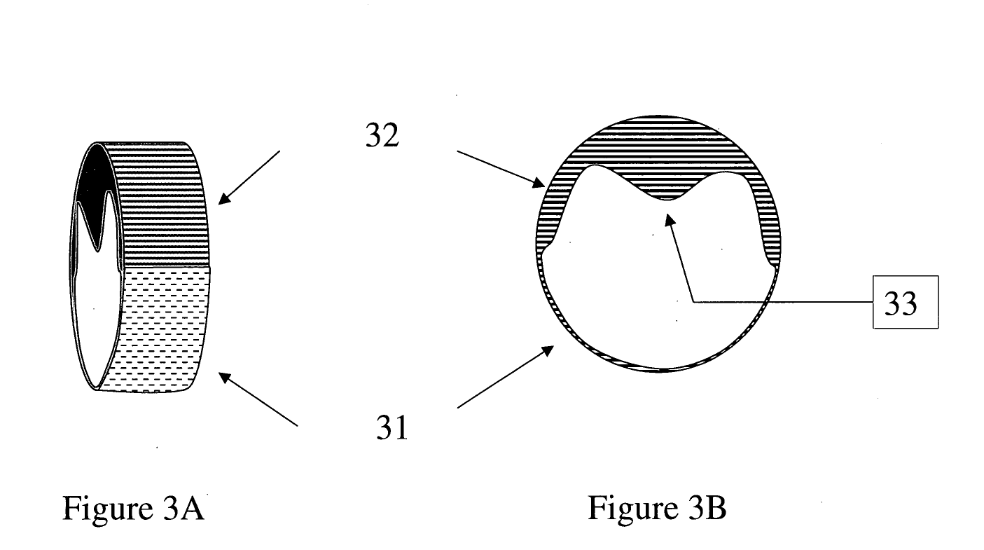

In certain embodiments, the annular structure may include regions of differing compliance. For example, as shown in FIGS. 3A and 3B, the annular structure includes a first, compliant region 31 and a second “strengthened” region 32, which second region is less compliant than the first region. The regions of varying compliance may be the result of a variety of different design parameters, e.g., having regions of differing thickness, having regions of differing materials, etc. In the representative embodiment shown in FIGS. 3A and 3B, the first compliant region has a thickness that is substantially less than the thickness of the second region, e.g., at least about 50-fold less, such as at least about 10-fold less. In these representative embodiments, the thickness of the first region may range from about 10 mm to about 0.1 mm, such as from about 20 mm to about 0.1 mm, including from about 10 mm to about 1 mm (e.g., from about 5 mm to about 1 mm), while the thickness of the second region may range from about 5 mm to about 0.1 mm, including from about 2 mm to about 0.1 mm. Also shown is protuberance 33.

The devices employed in the subject invention may be made up of rigid materials, flexible materials, or be a composite of both rigid and flexible materials. Whether the materials are flexible or rigid, they should be biocompatible for at least their intended use, such that they may be maintained in the body for the duration of the time that turbulence production in the vessel is desired. By biocompatible is meant that they should be capable of being maintained in an animal host for a period of time during which turbulence is to be produced with little or no, and preferably no, toxic effects for the animal host. Examples of suitable rigid biocompatible materials include, but are not limited to: medical grade alloys, such as cobalt-chromium alloy, titanium alloy, stainless steel, ceramics and composite materials, and the like. Examples of flexible materials include elastic materials, where suitable elastic materials are materials that exhibit elasticity at a relevant temperature range, i.e., below room temperature to body temperature, e.g., from about 10 to 50° C. at least, and are biocompatible. One type of biocompatible elastic material of interest is the class of memory alloys, including those described in: U.S. Pat. Nos. 5,876,434; 5,797,920; 5,782,896; 5,763,979; 5,562,641; 5,459,544; 5,415,660; 5,092,781; 4,984,581; the disclosures of which are herein incorporated by reference, e.g., biocompatible alloys that find use include those nickle-titanium (NiTi) shape memory alloys sold under the Nitinol™. Also of interest are polymeric materials, where representative polymeric materials of interest include, but are not limited to: biocompatible polymers and/or elastomers. Suitable biocompatible polymers include, but are not necessarily limited to, materials such as, for example, polyethylene, homopolymers and copolymers of vinyl acetate such as ethylene vinyl acetate copolymer, polyvinylchlorides, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyurethanes, polyvinylpyrrolidone, 2-pyrrolidone, polyacrylonitrile butadiene, polycarbonates, polyamides, fluoropolymers such as polytetrafluoroethylene and polyvinyl fluoride, polystyrenes, homopolymers and copolymers of styrene acrylonitrile, cellulose acetate, homopolymers and copolymers of acrylonitrile butadiene styrene, polyvinylchloride, silicone rubber, polymethylpentene, polysulfones, polyesters, polyimides, polyisobutylene, polymethylstyrene and other similar compounds known to those skilled in the art. Suitable, biocompatible elastomers include, but are not necessarily limited to, biocompatible elastomers such as medical grade silicone rubbers, polyvinyl chloride elastomers, polyolefin homopolymeric and copolymeric elastomers, urethane-based elastomers, and natural rubber or other synthetic rubbers, fluorinated polymers (e.g., PTFE), and the like. It should be understood that these possible biocompatible materials are included above for exemplary purposes and should not be construed as limiting.

The devices employed in the subject invention may or may not be size adjustable. As such, in certain embodiments, the devices may be adjustable so they can achieve annular structures of a variety of different dimensions, as desired. Alternatively, the devices may be designed so as to not be adjustable, such that they can only assume an annular structure of a single set of dimensions. The devices may also include a stabilizing element, e.g., lock, clasp or other type of element, which element maintains the annular structure of the device upon application around the vessel. In many embodiments, the devices will be configured to be open and closed, e.g., by means of a hinge or analogous structure, such that the device can be deployed about a target vascular location.

The above-described annular structures are, as indicated, merely representative the blood flow modulating devices that may be employed in the subject methods. For example, blood flow modulating devices that are not annular structure may also be employed. Non-annular structure blood flow modulating devices of interest include, but are not limited to: semi-circular structures, “horse-shoe” structures, crescent structures, etc. Furthermore, alternative types of blood-flow modulators may be employed. For example, vibratory elements that can transduce a remote signal into vibrational energy may be placed at a vascular site and activated in a manner that produces the desired blood flow modulation. Signal transducers of interest in such embodiments include sonic transducers, etc.

In certain embodiments, the devices partial annular or ring structures, such that in a deployed position they do not form a complete band about or around a target vessel. In such embodiments, the partial ring may be a structure that goes from a first to a second configuration upon deployment, such that prior to deployment it can easily be positioned about a target vessel and then following placement it securely closes around the vessel to provide the desired turbulence at the target location.

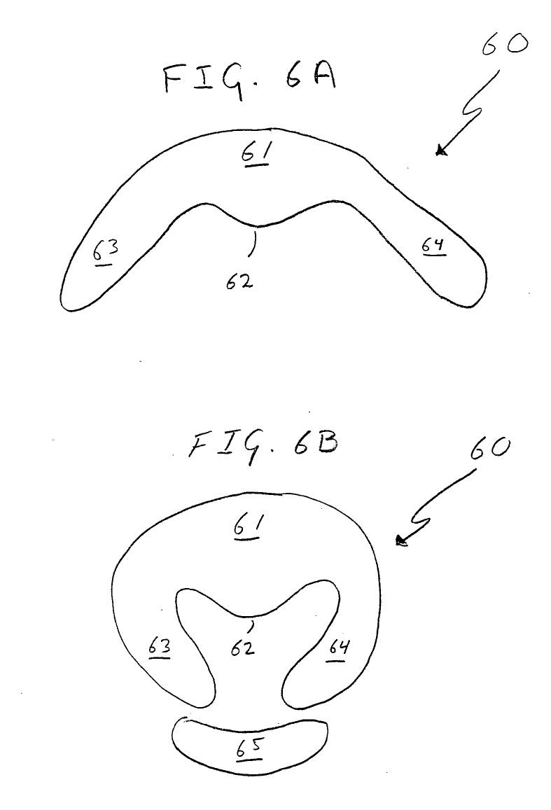

In certain of these embodiments, the device has a configuration in which a central portion that includes the at least one protuberance is flanked on one or both sides by an adjustable arm or grasping element. The arm may be shape adjustable such that it provides for the different configurations mentioned above. A representation of this embodiment is shown in FIGS. 6A and 6B. FIG. 6A shows device 60 having central domain 61 that includes protuberance 62 flanked by arm elements 63 and 64. Each of arm elements 63 and 64 is shape adjustable such that they can transition from an open position as shown in FIG. 6A to a closed position as shown in FIG. 6B.

Shape adjustable arm or clasp (63 and 64) elements can be provided in any convenient manner, e.g., by gas or fluid inflatable structures that, upon inflation adjust the arm from the first to second configuration; by use of phase reversible materials in the arm that transition from a first fluid state to a second rigid state in response to a stimulus (e.g., chemical, electromagnetic radiation, thermal, etc.) and thereby cause the arm to transition from the first to second configuration; via mechanically adjustable elements that cause the arm to transition from the first to second configuration (e.g., as provided by use of shape memory structures, with or without non-shape memory or removable rigid structures); etc. For example, a removable rigid element that maintains the arms in the first configuration may be coupled to the structure during placement, and then removed following placement. Upon removal of the rigid structure, shape memory elements present in the structure, e.g., in the arms, cause the arms to transition to the second configuration, such that the structure encloses about the target vessel. Alternatively, one can have two elements in an arm, where one of the elements changes length relative to the other and, in doing so, changes the configuration of the arm. Also shown in FIG. 6B is fastener element 65 which may be employed to secure the relationship of arms 63 and 64.

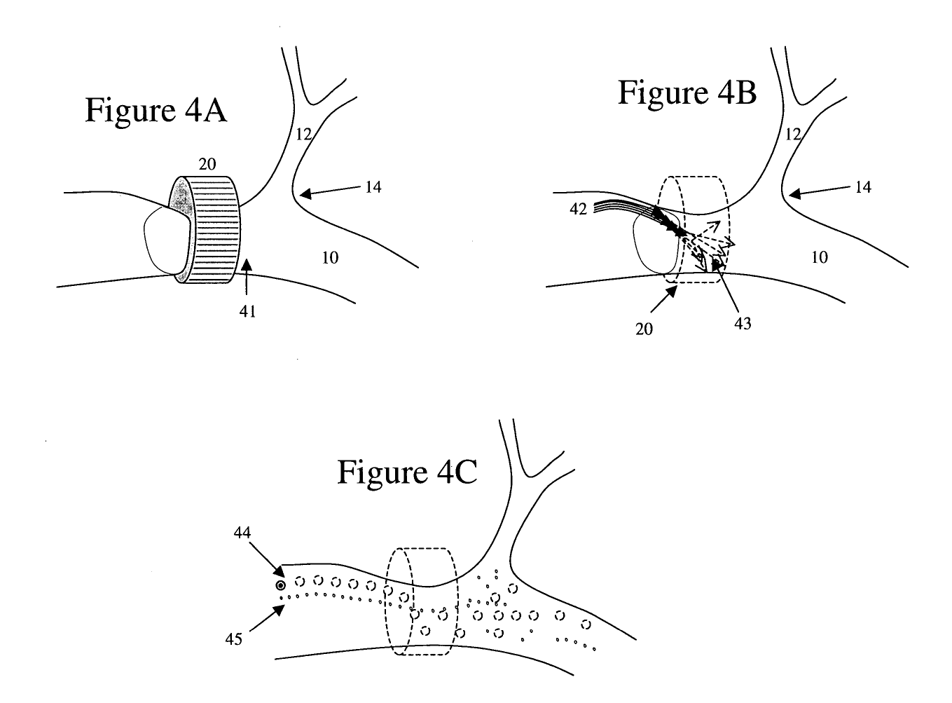

As indicated above, in practicing the subject methods the device is placed at an external location of the blood vessel in which the production of turbulence is desired. FIGS. 4A to 4C provide a depiction of placement of the representative device shown in FIG. 2 around an arterial vessel proximal to a branch point. In FIG. 4A, device 20 is positioned at site 41 which is proximal to branch point 14 of main blood vessel 10 and branch blood vessel 12. As indicated above, the distance of external target site 41 from branch point 14 may range from about 100 mm to about 1 mm, such as from about 50 mm to about 1 mm, including from about 30 mm to about 1 mm.

FIG. 4B provides a representation of the blood flow pattern and the production of turbulence therein that is achieved by placement of device 20 as shown in FIG. 4A. In FIG. 4B, prior to the region of the application, blood flow as a laminar flow pattern, as represented by lines 42. However, in the region encircled by device 20, the flow pattern becomes turbulent, as shown by the randomly directed arrows 43.

FIG. 4C shows how the production of turbulence as shown in FIG. 4B can reduce the risk of large particles from entering branch vessel 12 from main vessel 10. Prior to the region of the device 20, large particles 45 are found proximal to the luminal surface of main vessel 10, as these large particles, e.g., emboli, are found in the slowest moving layer of the laminar blood flow. Smaller particles 45 are found closer to the center of the blood vessel lumen, in the faster moving layers. The production of turbulence by device 20 causes particles 45 to flow closer to the cent of the lumen of the main vessel 10, and therefore not flow into the branch vessel 12.

In practicing the subject methods, the blood flow modulator device may be placed on or around the vessel of interest using any convenient protocol. As such, where the target vessel is directly accessible, e.g., during an open surgical procedure (such as in an open heart surgery, or an endoscopic procedure), the blood flow modulation device may be manually placed or positioned around the target vessel, which target vessel may have been pre-treated, e.g., to remove interfering tissue or structures, etc., as desired. Alternatively, a placement device which places or positions the blood flow modulator at the target site may be employed, e.g., which device may be analogous to placement devices known in the art, e.g., as disclosed in the patents cited above under the Relevant Literature heading. Alternatively, where minimally invasive procedures are employed, various percutaneous administration protocols/delivery devices may be employed, as desired.

In certain embodiments, the subject methods may include a blood flow evaluating step, in which blood flow patterns in the target vessel of interest are assessed and the resultant data obtained thereon is employed in the use of a blood flow modulating device, e.g., in determining the dimension of a particular device to be deployed, in determining the location at which the device is to be positioned, etc. In such methods, any convenient blood flow evaluating device/element may be employed, where representative such devices/elements include, but are not limited to: devices employing ultrasound, Doppler, magnetic technologies such as magnetic resonance imaging, and the like.

Where the obtained blood flow data is to be employed in the use of a particular blood flow modulator device, as described above, the data may be evaluated manually or automatically and employed to select the particular device and/or positioning of the device. Alternatively, automated protocols may be employed to select a device of particular dimensions and/or deployment location and provide these selections to the health care provider practicing the methods. For example, an algorithm or programming code can be employed in conjunction with a suitable processing element, e.g., computer, to make a selection of a device from a number of different dimensioned devices and/or to provide a target location for positioning of the device. Such programming can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information.

The above-described methods may be employed to produce turbulence in any vascular vessel, where in many embodiments the vessel is an arterial vessel. By arterial vessel is meant a vessel of a vascularized animal in which blood flows away from the heart. Generally the vascularized animals with which the subject invention is employed are “mammals” or “mammalian,” where these terms are used broadly to describe organisms which are within the class mammalian, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the animals or hosts, i.e., subjects (also referred to herein as patients), will be humans.

Utility

The subject invention finds use in any application where the production of turbulence in a vessel, e.g., an arterial vessel, is desired. One representative type of application in which the production of turbulence according to the subject invention finds use is in patients with arrhythmias, such as atrial fibrillation, that have a higher propensity to introduce potentially deleterious particulate matter into the vascular system. In this representative application, the subject methods and devices may be used to at least reduce the propensity or probability of large size particulate matter to enter a branch vessel from a main vessel. By large size particulate matter is mean particulate matter having a diameter of at least about 10 μm, such as at least about 100 μm, where the size range of large size particulate matter may range from about 10 μm to about 10 mm, such as from about 100 μm to about 3 mm. As such, the subject methods may be used to at least reduce the propensity of emboli present in blood to flow into a branch vessel from a main vessel, and in certain embodiments can substantially if not completely inhibit emboli or analogous particulate matter from entering a branch vessel from a main vessel. By at least reduce the propensity is meant that the probability that a given particle of large size, as described above, for entering a branch vessel from a main vessel is reduced by at least about 30%, including by at least about 75% for at least a subset of sizes of particles in the ranges described above.

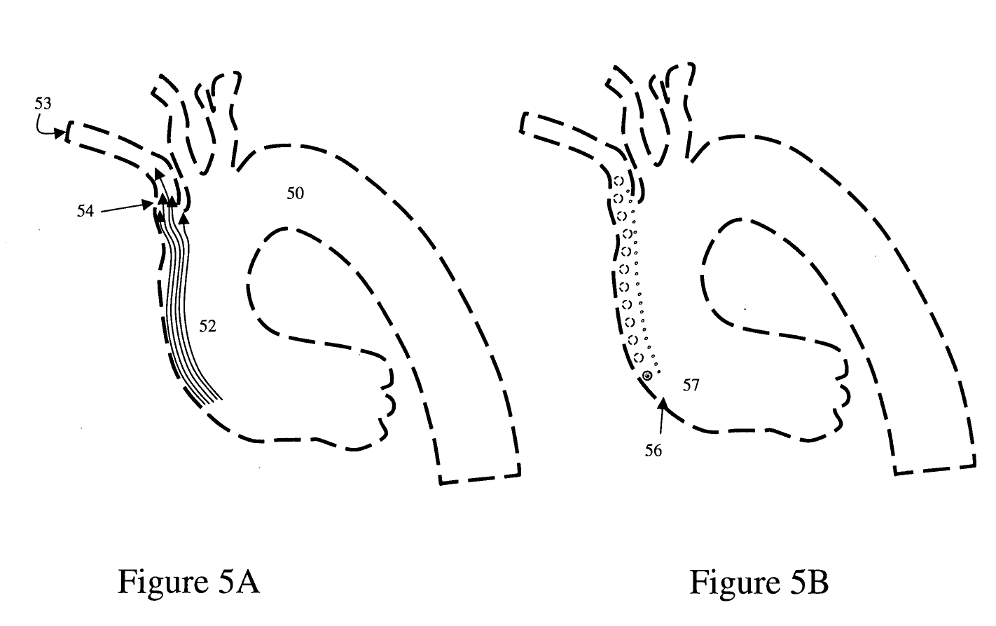

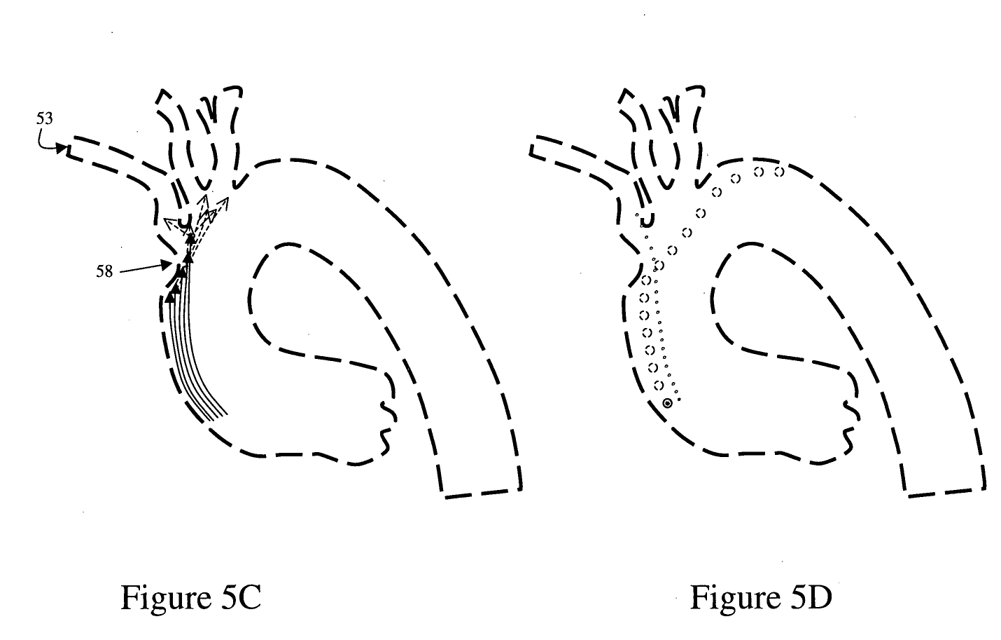

A specific representative application of interest in which the subject methods and devices find use is in conjunction with cardiac operations to reduce the risk of embolic byproducts from entering the brachiocephalic trunk from the aortic arch. As shown in FIG. 5A, blood flows through aortic arch 50 in a laminar flow pattern as shown collectively by lines 52, with the blood closest to the luminal surface of the aortic arch flowing into the brachiocephalic trunk 54, as represented by arrows 55. FIG. 5B provides a representation showing the flow of large particles 56 and small particles 57 through the same aortic arch. As can be seen by the flow path shown in the figure, because large particles 56 are found in the layer closest to the luminal surface, these particles tend to flow into the brachioencephalic trunk instead of staying in the aorta. FIG. 5C shows the impact of placing a blood flow modulator according to the present invention at branch point 58, which placement results in turbulence production and prevents large particles from entering the brachioencephalic trunk, as shown in FIG. 5D.

Systems

Also provided are systems for use in practicing the subject methods, where the systems at least include a blood flow modulatory device, as described above. The subject systems also may include a delivery element for delivering the device to a particular target site. Furthermore, the systems may include a blood flow evaluation element and/or a device selection element, which element provides a selection of a particular device and/or manner of using a device based on input blood flow data, as reviewed above. As used herein, the term “system” refers to a collection of elements that are brought together, e.g., from disparate sources, for a coordinated purpose.

Kits

Also provided are kits for use in practicing the subject methods, where the kits typically include one or more of the above blood flow modulation devices, as described above. In certain embodiments, the kits at least include two different blood flow modulation devices, where the devices may have different dimensions, so as to provide a selection of differently sized devices to the user during use. The kit may further include other components, e.g., delivery devices, blood flow evaluation elements, device selection elements, etc., as described above, which may find use in practicing the subject methods.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

It is evident from the above description that the subject invention provides an important new way to reduce the risk of particulate matter, e.g., emboli, from entering branch vessels during vascular procedures. Accordingly, the present invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of producing turbulence in blood flow at a target site of a blood vessel, said method comprising:
positioning a blood flow modulator on an external site of said blood vessel at least proximal to a branched vascular site in a manner sufficient to produce turbulence in blood flow at said target site.

2. The method according to claim 1, where said blood vessel is an artery.

3. The method according to claim 1, wherein said blood flow modulator changes the internal surface of said blood vessel in a manner sufficient to produce said turbulence.

4. The method according to claim 3, wherein said blood flow modulator is an annular device that is positioned around said blood vessel.

5. The method according to claim 4, wherein said annular device comprises an internal protrusion configured to cause said change in said internal surface of said blood vessel.

6. The method according to claim 5, wherein said annular device comprises regions of different compliance.

7. The method according to claim 1, wherein said method further comprises evaluating blood flow in said vessel prior to positioning said blood flow modulator.

8. The method according to claim 7, wherein said method further comprises selecting a blood flow modulator of a particular configuration based on said evaluated blood flow.

9. The method according to claim 1, wherein said method further comprises removing said blood flow modulator following producing of said turbulence.

10. The method according to claim 1, wherein said method is a method of at least reducing the propensity of particles in said blood from entering a branch vessel.

11. The method according to claim 10, wherein said particles are emboli.

12. A method of at least reducing the propensity of emboli to enter a branch blood vessel from a main blood vessel, said method comprising:
positioning a blood flow modulator on an external site of said main blood vessel at least proximal to said branch blood vessel in a manner sufficient to produce turbulence in blood flow in said main blood vessel to at least reduce the propensity of emboli to enter to branch blood vessel.

13-19. (canceled)

20. A blood flow modulation device that can be positioned around an external surface of a blood vessel to produce an annular structure that has a protuberance of sufficient dimensions to produce turbulence in blood flow in a blood vessel around which said device is positioned.

21. The blood flow modulation device according to claim 20, wherein said annular structure comprises regions of differing compliance.

22. The blood flow modulation device according to claim 21, wherein said regions of differing compliance have different thicknesses.

23. The blood flow modulation device according to claim 20, wherein said annular structure has an outer diameter that ranges from about 1.0 mm to about 70 mm.

24. The blood flow modulation device according to claim 23, wherein said annular structure as a width that ranges from about 0.5 mm to about 65 mm.

25. The blood flow modulation device according to claim 24, wherein said device includes a stabilizing element to maintain said annular structure upon positioning of said device around a blood vessel.

26. A kit comprising:
a blood flow modulation device according to claim 20; and
instructions for employing said device to produce turbulence in a blood vessel.

27-31. (canceled)

32. A system for producing turbulence in a blood vessel, said system comprising:
(a) at least one blood flow modulator according to claim 20; and
(b) a device for positioning said blood flow modulator around a blood vessel.

33-34. (canceled)
